(19) United States
(12) Patent Application Publication
(10) Pub. No.: US 2026/0345081 A1
(43) Pub. Date: Nov. 5, 2026

(54) Modular sensing and actuating layered substrate for electronic catheters

(71) Applicant: Berne University of Applied Sciences, Biel (CH)

(72) Inventors: Nishant Gupta, Nidau (CH); Gerhard Kuert, Magglingen (CH); Thomas Niederhauser, Oberwangen b. Bern (CH)

(73) Assignee: Berne University of Applied Sciences, Biel (CH)

(21) Appl. No.: 19/158,942

(22) PCT Filed: Feb. 22, 2024
(86) PCT No.: PCT/EP2024/054554

§ 371 Date: Aug. 22, 2025

(30) Foreign Application Priority Data
Feb. 23, 2023 (EP) EP20230158156

(51) Int. Cl. A61B 5/00; H05K 1/11; H05K 1/14; H05K 1/189; H05K 3/36

(52) U.S. Cl. CPC A61B 5/6852; H05K 1/118; H05K 1/147; H05K 1/189; H05K 3/363; A61B 2562/12; A61B 2562/166; A61B 2562/222; H05K 2201/058; H05K 2201/10151

(57) ABSTRACT

A modular sensing and actuating layered substrate for electronic catheters including two or more of one or more active modules having a first and a second connector segment, and at least one node including one or more transducers, and optionally one or more spacer modules having a first and a second connector segment, but no node nor sensor. The first connector segments are electrically connected to the second connector segment of the adjacent module. The modules, the connector segments, and the nodes are based on a flexible printed circuit board FPCB, and each connector segment includes multiple of electrically conductive contact pads. When the first connector segment is electrically connected to the second connector segments of another module, the contact pads of the first connector segment are in contact with the contact pads of the second connector segment. Also is a process to manufacture an electronic catheter having the substrate.

### TECHNICAL FIELD

The present invention relates to a modular sensing and actuating layered substrate for electronic catheters, and a process to manufacture an electronic catheter comprising the modular layered substrate.

### BACKGROUND OF THE INVENTION

Catheters have become in modern medicine more and more important and are used today in many applications. In recent times, improved catheters allow access to several internal organs for acquiring multiple physiological signals needed for patient monitoring, diagnostic or therapeutic purposes. Continued miniaturization allows the addition of integrated circuits (ICs) and transducers in combination with flexible printed circuit board (FPCB) technology, which enables low-cost fabrication of flexible catheters with an electronically functional surface, as demonstrated by E. Kuert et al. in “Efficient Thermobonding Process Forming a Polyurethane Based Diagnostic Catheter with Liquid Crystal Polymer”, Annu. Int. Conf. IEEE Eng. Med. Biol. Soc., vol. 2019, pp. 6163-6166, July 2019. Applications which otherwise rely on skin-attached sensors may benefit from improved signal quality by integrating multiple sensor modalities, e.g., bio-potentials, pressure, oximetry, temperature, etc., at suitable positions (nodes) along the length of catheters, as shown by C. Bürgin et al., “Multichannel esophageal signals to monitor respiratory rate in preterm infants”, Pediatr. Res., vol. 91, no. 3, pp. 572-580, February 2022, and by N. Gupta et al., “A Surface-integrated sensor network for personalized multifunctional catheters*,” Ann u. Int. Conf. IEEE Eng. Med. Biol. Soc., vol. 2023, pp. 1-4, July 2023.”.

The large number of different catheter applications, combined with the various needs of different people, e.g., in size, length, and purposes, result in the requirement—and thus in the production—of a high number of different catheters. This increases—among others—the complexity in logistics, costs due to the relatively small numbers of individual catheters made. Furthermore, the present means of production makes it in practice impossible to use highly specific, individual, and custom-made catheters.

Therefore, there is a need to provide means for simplified production to reduce complexity, logistics, costs, as well as enable with reasonable effort to obtain specific, custom-made, and individual catheters at reasonable costs.

### SUMMARY OF THE INVEBNTION

Surprisingly, it was found that the problem can be solved with a modular sensing and actuating layered substrate (1) for electronic catheters (2), comprising a total of two or more of:
one or more active modules (4), each module (4) comprising a first (41) and a second (42) connector segment, and at least one node (43) comprising one or more transducers (44), and
optionally one or more spacer modules (5), each module (5) comprising a first (51) and a second (52) connector segment, but no node nor sensor,
wherein the first connector segments (41, 51) of the active (4) and the optional spacer (5) modules are electrically connected to the second connector segment (42, 52) of the adjacent active (4) or spacer (5) module, characterized in that
the modules (4, 5) with the connector segments (41, 42, 51, 52), and the nodes (43) are based on a flexible printed circuit board FPCB (8), and
each connector segment (41, 42, 51, 52) comprises on at least one flat, i.e., large, surface of the FPCB (8) a multiple of electrically conductive contact pads (81), wherein, when the first connector segment (41, 51) of one module (4, 5) is electrically connected via an electrical connection (6) to the second connector segments (42, 52) of another module (4, 5), the contact pads (81) of the first connector segment (41, 51) are in contact with the contact pads (81) of the second connector segment (42, 52).

Claimed also is a process to manufacture an electronic catheter (2) comprising the modular layered substrate (1) according to the invention, comprising:
cutting out of a pre-shaped FPCB sheet a total of two or more of one or more active modules (4) comprising a first (41) and second (42) connector segment, and at least one node (43), optionally one or more spacer modules (5) comprising a first (51) and second (52) connector segment, and optionally the distal module (3) comprising the connector segment (31), and optionally at least one node (33);
mounting on each node (33, 43) of the one or more active modules (4), and of the optional distal module (3), the transducers (34, 44) onto at least one surface of the FPCB (8) and optionally the activatable transducer interface (36, 46) and an activation logic circuit comprising preferably the D-type flip-flop (DFF; 35, 45);
connecting the first connector segment (41, 51) of the active (4) and the optional spacer (5) modules with the second connector segment (42, 52) of the adjacent active (4) or spacer (5) module via a flat, i.e., large, surface of the connector segments (31, 41, 42, 51, 52), thus forming the substrate (1) of at least one the claims 1 to 11;
wrapping the obtained substrate (1) around a catheter tube or cable (2a) and/or inserting the substrate (1) into the catheter tube (2a), wherein the substrate (1) is adhered to the catheter tube or cable (2a) to obtain the electronic catheter (2).

The modular sensing and actuating layered substrate (1) according to the invention, and the process to manufacture an electronic catheter (2) comprising said modular layered substrate (1) possess unexpectedly a large number of advantages.

### BRIEF DESCRIPTION OF THE DRAWINGS

FIG. 1 discloses a non-limiting example of the active module, which includes a first and a second connector segment, and at least one node (43);

FIG. 2 discloses a non-limiting example of an optional spacer module;

FIG. 3a shows a first connector segment of either the optional distal module, the active module or the optional spacer module and a second connector segment of either the active module or the optional spacer module;

FIG. 3b shows the first connector segment connected to the second connector segment Via one of their flat, i.e., large, surfaces, i.e., the surface having the larger extension;

FIG. 3c shows a cross section of the first connector segment connected to the second connector segment of the connected modules as shown in FIG. 3b.

FIG. 4 presents the various steps from individual modules to the assembled substrate being wrapped around a catheter tube or cable to the manufactured electronic catheter.

FIG. 4a shows specifically—from right to left—the optional distal module with one connector segment and an optional detachable segment for performance test;

FIG. 4b shows the electrically connected modules of FIG. 4a forming the substrate according to the invention;

FIG. 4c shows an example of a catheter tube with an optional hole towards to proximal, i.e., left, end of the tube to allow a connector segment to pass from the outer surface of the tube into the inner area of the tube;

FIG. 4d shows the finally assembled electronic catheter having the substrate of FIG. 4b with the distal module, a spacer module, an active module, and another spacer module leading towards the external controller;

FIG. 5a shows a schematic cross-section representing various possible scenarios of sections of the electronic catheter with a portion of the FPCB of a node of either the distal or active module;

FIG. 5b shows—similar to FIG. 5a—a schematic cross-section representing various possible scenarios of sections of the electronic catheter with a portion of the same FPCB of a node of either the distal or active module.

FIG. 6 presents a non-limiting example of a schematic section of the substrate comprising two active modules being connected to each other via a spacer module; and

FIG. 7 presents an exemplary, non-limiting activation logic circuit with two nodes at positions n and n+1, respectively, of two active modules being connected to each other via a spacer module.

### DETAILED DESCRIPTION OF THE INVENTION

The fact that the modular sensing and actuating layered substrate (1), i.e., the substrate (1), is based on a number of different modules (4, 5), allows the production of separate individual modules (4, 5) in large numbers. Said prefabricated modules (4, 5) can then be assembled based on the specific needs of a patient and thus—basically in any combination—to highly specific, customized, and individual modular substrates (1). Hence, modules which share the same connection interface can be placed at specific places of the catheter, allowing to repeat easily the sensing functionality at multiple locations of the catheter. Since each module allows multiple sensor types, a large number of, e.g., 100 or more, sensing zones in a single substrate (1) for monitoring e.g., bio-potentials, temperature, flow, oxygen saturation, pressure, and many more parameters is possible. Said customized substrates (1) can then be applied onto the inner or outer surface of the catheter tube or cable (2a) to obtain the electronic catheter (2) according to the invention, which itself is therefore customizable. Hence, modular sensing and actuating layered substrate (1) according to the invention allows to reduce significantly the design effort for new substrates for catheters.

The one or more active modules (4) may be of the same or of a different kind, i.e., they may e.g., differ in size, in number of nodes (43), type and/or number of transducers (44). And the optional, one or more spacer modules (5) may vary e.g., in size, and in particular in its length. Said modules (4) may be combined with each other and optionally with one or more spacer modules (5) resulting in a huge number of possible combinations.

Furthermore, when a substrate (1) comprises e.g., two or more different active modules (4), i.e., active modules (4) may have a different architecture and e.g., a different number and/or type of transducers (44), the order of the modules (4) within a substrate (1) can easily be changed upon assembling the desired modules (3, 4, 5) in the customized order to obtain the individually designed and desired substrate (1). Thus, no separate substrate (1) needs to be made when e.g., the order of just two modules (4) needs to be changed.

Due to the fact that the contact pads (81) of the first connector segments (41, 51) are in contact with the contact pads (81) of the second connector segments (42, 52) of the active (4) and spacer (5) modules allows a random combination of the modules (4, 5). Hence, the modules (4, 5) are fully interchangeable.

The fact that—within the substrate (1)—the contact pads (81) of the first connector segments (31, 41, 51) are in contact with the contact pads (81) of a second connector segment (42, 52) of the active (4) and spacer (5) modules allows daisy-chaining of the modules (4, 5). Furthermore, the use of the activation logic circuit comprising preferably the D-type flip-flop (DFF; 35, 45) and the activatable, i.e., selectable, transducer interface (36, 46) allows daisy-chaining of a random number and in any combination of the modules (3, 4) without altering the number or the nature of signals associated with the contact pads (81). Thus, the modular sensing and actuating layered substrate (1) according to the invention allows signal amplification and multiplexing of the recorded and transferred signals—even from one to adjacent modules via the connector segments (42, 52)—and thus even from the distal end of the substrate (1) to the external control unit (7). Therefore, the number of internal and external connections required is significantly reduced at both, the connector segments (42, 52) as well at the interface to the external control unit (7).

Hence, the order of the modules (4) within a substrate (1) can easily be changed upon assembling the desired modules (3, 4, 5) in the customized order to obtain the individually designed and desired substrate (1). Thus, no separate substrate (1) needs to be made when e.g., the order of just two modules (4) needs to be changed or the customization needs can be met by increasing the number of prefabricated modules. With other words, the connectors are designed to allow physical daisy-chaining of the modules (3, 4). And the activation circuit ensures that basically an indefinite, i.e., a very large, number of modules in any combination can be daisy-chained without altering the design, i.e., architecture, of the module and/or connector. Therefore, a large number of different customization needs can be addressed with a small number of designed modules (3, 4) and assembled substrates (1).

A key element of the substrate (1) according to the invention is the flexible printed circuit board FPCB (8), the substrate (1) is based on, wherein the FPCB (8) provides a number of high beneficial advantages, which can be achieved with a substrate (1) being based on FPCB (8) only. Thus, the FPCB (8)—being a layered composite material comprising, among others, one or more dielectric layers, one or more signal layers, and being covered by a dielectric coating—provides a high flexibility of the substrate (1), which allows that the latter can be easily wrapped around a catheter tube or cable (2a), even of small diameter. Furthermore, the high flexibility of the FPCB (8) permits the substrate (1) to easily participate in any bending of the catheter (2). Due to the minor thickness of the FPCB (8)—and thus of the modules (4, 5) and therefore of the substrate (1)—of e.g., only about 0.05 to 4 mm, the outer diameter of catheter tube or cable (2a) extends only marginally when the substrate (1) is being wrapped around it. In addition, the various materials of the FPCB (8) impart a good biocompatibility, i.e., the FPCB (8) does not have any adverse effects to the human body. Since the active module (4) is based on FPCB (8), a large number of various transducers (44)—either the same type at different positions and/or different transducers (44) at about the same position—can be placed onto one and the same module (4) which is based on FPCB (8). Furthermore, it was surprisingly found that when the substrate (1)—and thus of the modules (4, 5) and the connector segments (41, 42, 51, 52)—is based on FPCB (8), a large number of signals from the various transducers (44) can be transmitted easily and without delay.

It is noted that EP-A-3955710 discloses a catheter which comprises a catheter tube and a flexible printed circuit board (FPCB), wherein the FPCB covers essentially the catheter circumference for predetermined FPCB transducer segment length, especially at the distal portion, wherein the FPCB in the FPCB transducer segment comprises a scaffold structure with a plurality of FPCB free spaces and one FPCB free surface portion creating maximum mechanical stability with the least amount of material and a FPCB scaffold structure which ensures maximum flexibility. The FPCB comprises transducer patches which are connected by traces on the straight scaffolds of the scaffold structure. The FPCB free spaces are configured for preventing, circumventing and/or compensating the kinking behavior of the catheter tube. A modular sensing and actuating layered substrate based on active and spacer modules which are electrically connected to each other, and an electronic catheter comprising such a substrate, wherein said substrate and said catheter are easy customizable based on patient's needs, are not disclosed.

US-A-2010/084160 refers to a cable assembly for interconnecting a plurality of circuit boards together by using a connector assembly connected to each of the circuit boards. The cable assembly includes a first cable having a first end part and a second cable having a second end part. A first periphery of the first end part has a plurality of first half vias that collectively form a column along a width direction of the connector assembly. A second periphery of the second end part has a plurality of second half vias that collectively form a column along the width direction of the connector assembly. The first and second end parts are coupled together to form a connecting unit, such that the first half vias and the second half vias are joined together to form full vias. The connector assembly may be used for a high-density connection between electronic devices. Hence, the cables connect circuit boards, but the cables themselves are not based on flexible printed circuit boards, nor are intended for providing a sensing or actuating functionality, such as in catheters. Furthermore, the coupling of the first and second parts occurs at an edge of the connecting parts, resulting in a split line. Thus, the connecting unit requires a further element, i.e., a connector assembly, onto which the connecting parts are assembled. Such an arrangement is, however, not suitable for the claimed modular sensing and actuating layered substrate for use in catheters, since such an edge-type connection allows only a restricted number of connections and increases the size of the substrate. In addition, connecting cables relates to a distinct different technology than connecting FPCB-layers, as disclosed by the present invention.

### The Substrate (1)

The substrate (1) according to the invention is a modular sensing and actuating layered substrate (1) for electronic catheters (2). Thus, the substrate (1) is flexible enough to be wrapped easily around a catheter tube or cable (2a), i.e., onto the outer surface of the catheter tube (2a) or can be inserted into a catheter tube (2a) and placed, i.e., onto the inner surface of the catheter tube (2a), to result in the customized electronic catheter (2) obtained according to the process of the invention.

The term sensing substrate stands for a substrate (1) which comprises transducers (34, 44) which sense, i.e., observe, physiological stimuli or conditions, such as electrocardiogramyography (EMG), impedance, temperature, pressure, and/or oxygen saturation. Such transducers (34, 44) may be active, i.e., convert, in particular directly convert, external stimuli to a measurable voltage or current, or passive, i.e., require additional power or amplifier to generate a measurable voltage or current. And the term actuating substrate stands typically for a substrate (1) which comprises actuators, i.e., said transducers capable of inducing a biological action or stimulation, e.g., nerve activation.

The external control unit (7) is a separate device designed and fabricated using techniques known in the art. Thus, the skilled person in the art is well capable of making a suitable control unit (7) based on existing control units which are commercially available and dedicated for electronic catheters of the prior art. The unit (7) may be capable of controlling the transducer interfaces (36, 46) and further processing the signals, i.e., data, which are acquired by the transducers (34, 44) of the substrate (1) and sent to the control unit (7). Furthermore, the control unit (7) may also act as power source for the transducers (34, 44) of the substrate (1), including for performing of an action on the, e.g., passive, transducers, and actuators.

The substrate (1) comprises a total of two or more modules (4, 5), i.e., either at least two active modules (4) or at least one active module (4) and one spacer module (5). Optionally, a distal module (3) comprising one connector segment (31) may form the closure of the distal end of the substrate (1). The modules (3, 4, 5) are connected to one another via the connector segments (31, 41, 42, 51, 52) to allow unobstructed transmission of the electric signals from one module to one or more other modules.

The connector segments (31, 41, 42, 51, 52) are connected to one another via their flat, i.e., large, surfaces, i.e., the upper or lower side of the connector segments (31, 41, 42, 51, 52) and thus to the surface having the larger extension, and not via an edge side of the connector segments, i.e., an edge-type connection. This connection type provides sufficient stability and allows the connection of a large number of signal paths (83x, 86x) from one module (3, 4, 5) to another module (4, 5) without the requirement of a connector assembly, i.e., an additional element. Thus, the thus connected modular sensing and actuating layered substrate (1) is particularly suited for electronic catheters (2), which have inherently limited space.

### The Modules (3, 4, 5)

The term module (3, 4, 5) is—according to the present invention—understood to be an element which is manufactured separately and thus being an individual part for assembling the substrate (1) of the invention. The modules (3, 4, 5) are based on the flexible printed circuit board FPCB (8). Most preferably, each module (3, 4, 5) comprises the same number of contact pads (81), and thus the same number of electrical connections, wherein the number of contact pads (81) differs most typically from the number of modules (3, 4, 5).

The one or more active modules (4) comprise a first (41) and a second (42) connector segment, and at least one node (43) comprising one or more transducers (44).

The optional one or more spacer modules (5) comprise a first (51) and a second (52) connector segment, but no node nor transducer. Hence, the spacer modules (5) cannot acquire biosignals or perform an action via an active transducer. The primary function of spacer module (5) is to bridge the regions where acquisition of biosignals and/or physiological activation is not required. However, the spacer modules (5) may also comprise one or more integrated circuits (IC) for signal conditioning, communication, and/or an antenna or energy storage such as a battery or supercapacitors.

The first connector segments (41, 51) of the active (4) and the optional spacer (5) modules are electrically connected to the second connector segment (42, 52) of the adjacent active (4) or spacer (5) module.

According to the present invention,
the modules (4, 5) with the connector segments (41, 42, 51, 52), and the nodes (43) are based on a flexible printed circuit board FPCB (8), and
each connector segment (41, 42, 51, 52) comprises on at least one flat, i.e., large, surface of the FPCB (8) a multiple of electrically conductive contact pads (81), wherein, when the first connector segment (41, 51) of one module (4, 5) is electrically connected via the electrical connection (6) to the second connector segments (42, 52) of another module (4, 5), the contact pads (81) of the first connector segment (41, 51) are in contact with the contact pads (81) of the second connector segment (42, 52). Most preferably, all contact pads (81) of the first connector segments (41, 51) are in contact with all contact pads (81) of the second connector segments (42, 52). Thus, the first connector segments (41, 51) may be identical to the second connector segments (42, 52) and designed to connect to each other easily. Alternatively, the first connector segments (41, 51) and the second connector segments (42, 52) are designed e.g., in a plug and outlet mode.

In one particularly preferred embodiment, the substrate (1) further comprises a distal module (3) comprising one connector segment (31), and preferably at least one node (33) comprising one or more transducers (34), wherein the distal module (3) with the connector segment (31), and the nodes (33) are based on the flexible printed circuit board FPCB (8). Said distal module (3) with the only one connector segment (31) forms the head segment of the substrate (1), i.e., is finally placed at the head of the catheter (2) which will be introduced into the body first. Thus, when the distal module (3) comprises one or more nodes (33) with one or more transducers (34), the distal module (3) is similar to the active module (4) but comprises one connector segment (31) only. Alternatively, when the distal module (3) comprises neither a node (33) nor a transducer (34), the distal module (3) is similar to the spacer module (5) but comprises one connector segment (31) only. Thus, the distal module (3), if present, terminates the sequence of two or more modules (4, 5) and the assembled substrate (1) does not have a connector segment (42, 52) not being connected to another connector segment (41, 51). As such, the distal module (3) is opposite to the module (4, 5) being closest towards the external control unit (7).

In one preferred embodiment,
the active module (4) is connected to one or two other active modules (4), to one or two spacer modules (5), and/or to the distal module (3),
the spacer module (5) is connected to one or two active modules (4), and/or to the distal module (3), and/or
the distal module (3) is connected to the active module (4) or the spacer module (5),
wherein the connector segment (41, 51) of the module (4, 5) which is furthest away from the distal module (3), or from the most opposite connector segment (42, 52), is configured to be connected to the external control unit (7).

Thus, the substrate (1) may compose of one or more of the following exemplary sequences, wherein (4) stands for one active module (4), (5) stands for one spacer module (5), and (7) stands for the external control unit (7):
4-4→7
4-5→7
4-5-4→7
3-4-5-4→7
3-5-4-5→7
3-4-4→7
3-4-5→7
3-4-5-5→7
5-4-5-4→7

The above scheme demonstrates the high flexibility in assembling the substrate (1). Hence, the latter is highly customizable according to individual needs.

Hence, the optional distal module (3), the one or more active modules (4) and the optional one or more spacer modules (5) are most preferably connected to form a longitudinal substrate (1) which is capable to be wrapped around the catheter (2), wherein the connector segment (41, 51) of the module (4, 5), which is furthest away from the distal module (3), or from the most opposite connector segment (42, 52), is configured to be connected to an external control unit (7). Thus, in case there is no distal module, the most distal connector segment (42) would have no connection. Furthermore, the longitudinal shape of the substrate (1) provides an optimal form to be wrapped around a catheter tube or cable (2a) and/or inserting the substrate (1) into the catheter tube (2a) to form the catheter according to the process of the invention.

The module (4, 5), which is furthest away from the distal module (3), or from the most opposite connector segment (42, 52), is also called proximal module. This proximal module is the module (4, 5), which is closest to the control unit (7) and thus it can be either an active module (4) or a spacer module (5).

### The Connector Segments (31, 41, 42, 51, 52) and Contact Pads (81)

The term connector segment is—according to the present invention—a segment of a module (3, 4, 5) which is capable of electrically connect with another connector segment of another module (4, 5). Hence, electrical signals can be transported unobstructed from one module to one or more other modules.

The first (41) and second (42) connector segments of a modules (4, 5) are most typically arranged on both longitudinal ends of the modules (4, 5). Hence, the connector segments (41, 42) are positioned opposite to each other.

Most preferably, the connector segments (31, 41, 42, 51, 52) of the distal (3), active (4) and spacer (5) modules are identical in terms of number and location of contact pads (81) to allow each module (3, 4, 5) to be daisy-chained to any other module (4, 5) and to allow a smooth connection of the connector segments (31, 41, 42, 51, 52) to one another, wherein at least one of the modules (3, 4, 5) may be interfaced by the external control unit (7) via the same electrical connections (6) as the module (4, 5) which is located most proximally to the external control unit (7).

The number of contact pads (81) per connector segment (31, 41, 42, 51, 52) may be small, e.g., 4 to 10, but can increase to e.g., 20 to 40, or even higher.

The architecture of the contact pads (81) may be—and preferably is—identical on each connector segment (31, 41, 42, 51, 52) to avoid any incompatibility between different connector segments (31, 41, 42, 51, 52). In this embodiment, one preferred architecture possesses a flat surface. Thus, when two connector segments are in contact with each other, the flat surfaces of the contact pads (81) are opposite to each other with at least partial contact to allow good signal transmittance. Alternatively, the contact pads (81) of the connector segments (31, 41, 51) may be of one kind and the connector segments (42, 52) of another kind, wherein the connector segments (31, 41, 51) and the connector segments (42, 52) are in the plug and outlet mode. This enhances connectivity of the adjacent contact pads (81) and thus of the adjoining connector segments (31, 41, 42, 51, 52). Hence, the first connector segment (31, 41, 51) is electrically connected to the second connector segment (42, 52) via an electrical connection (6).

In order to facilitate to join one connector segment (31, 41, 51) to the adjoining connector segment (42, 52), it is often preferred when the contact pads (81) of the first connector segment (31, 41, 51) are oriented in one direction, while the contact pads (81) of the second connector segments (42, 52) are oriented to the opposite side.

In one preferred embodiment, the electrical connection (6) between the first connector segment (31, 41, 51) of the modules (3, 4, 5) and the second connector segment (42, 52) of the adjacent module (4, 5) is a connection
between at least one of the contact pads (81) of the first connector segment (31, 41, 51) and at least one of the contact pads (81) of the second connector segment (42, 52),
between the area besides to the contact pads (81) of the first connector segment (31, 41, 51) and the area besides to the contact pads (81) of the second connector segment (42, 52), and/or'
obtained by a layer which surrounds the first connector segment (31, 41, 51) and the adjacent second connector segment (42, 52),
wherein
the connection i) is preferably a solder, in particular a lead-free solder alloy such as Sn96.5Ag3Cu0.5; an anisotropic conductive film (ACF), an anisotropic conductive paste (ACP), an electrically conductive adhesive, wherein said adhesive is preferably an isotropic or anisotropic adhesive; or a welding, preferably obtained by thermo-sonic compression. Thus, the electric current is conducted from a contact pad (81) from one connector segment via the connection i) to the contact pad (81) of the adjacent connector segment;
the connection ii) is preferably a mechanical engagement; an engagement aided by magnets or a magnet and a magnetizable material; or based on an adhesive. Thus, the electric current is conducted from a contact pad (81) from one connector segment preferably direct to the adjoining contact pad (81) of the adjacent connector segment, while the two adjacent connector segments (31, 41, 51; 42, 52) are fixed together by the connection ii); and/or
the layer of the connection iii) is preferably obtained by molding, spraying, wrapping and/or encasing the first and second connector segments (31, 41, 42, 51, 52), wherein said layer is preferably based on a non-conductive material, which are known to the skilled person in the art. Thus, the electric current is conducted from a contact pad (81) from one connector segment preferably direct to the adjoining contact pad (81) of the adjacent connector segment, while the two adjacent connector segments (31, 41, 51; 42, 52) are fixed together by the connection iii).

This provides a good and permanent electrical connection between the pads (81) which maintains flexibility of the substrate (1) required to form the catheter (2). Furthermore, conducting the electrical current in a most optimal manner is maintained.

### The Nodes (33, 43)

The term node (33, 43) is—according to the present invention—understood to be a segment of the active (4) and the optional distal (3) module, wherein said segments may be activated and/or controlled independent from other nodes (33, 43) of the same module (3, 4). In many cases it is preferred that the nodes (33, 43) possess a broadened shape, i.e., width—in comparison to the standard width of the module (3, 4, 5)—to allow placing the transducers at several places along the periphery of the catheter for sensing/actuating in multiple directions and optionally further components onto the node (33, 43). If the modules (3, 4) comprise two or more nodes (33, 43), the nodes (33, 43) with their broadened shape may be connected to the adjacent node or nodes by a narrower width of the FPCB, i.e., a narrowed width compared to the width of the node (33, 43). The standard width of the FPCB relates most typically to about the width of the spacer module (5) and/or the connector segments (31, 41, 42). Hence, this shape provides a scaffold pattern of the FPCB (8), which improves flexibility.

In a preferred embodiment,
the one or more active modules (4) and/or the optional distal module (3) of the substrate (1) comprise at least 1, preferably 2 to 50, in particular 2 to 20, nodes (33. 43), wherein, when the substrate (1) comprises a plurality of modules (4), the number of nodes (43) of each module (4) may be different, and the number and type of transducers (44) on each node (43) may be the same or different, which increases the flexibility—and thus the customizability—of the substrate (1) even more; and/or
the shape of the one or more active modules (4), of the optional one or more spacer modules (5), and of the optional distal module (3) of the substrate (1) exhibit a linear shape, a zig-zag shape, U-shape, Ω-shape, i.e., Omega-shape, a meandering form, and/or is based on a polygonal, e.g., hexagonal, honeycomb or scaffolding shape, as disclosed in EP-A-3955710. The non-linear shape increases the flexibility, such as bending capability, of the substrate (1) and reduces irregularities or breakage of the substrate (1) upon bending the catheter (2) comprising the substrate (1).

In another preferred embodiment, at least one of the modules (3, 4, 5) may contain one or more additional connection pads for testing the interconnected modules in parallel during the manufacturing process. Said additional connection pads may be physically detached, e.g., by cutting, at a suitable stage of the making of the catheter (2), in particular when making layered substrate (1) and thus before joining the substrate (1) with the catheter tube (or cable (2a).

In another particular preferred embodiment,
each node (33, 43) of the distal (3) and active (4) modules comprises one or more transducers (34, 44), each with an activatable, i.e., a selectable, transducer interface (36, 46) and an activation logic circuit comprising preferably one or more, in particular only one, D-type flip-flop (DFF, 35, 45) for activating/deactivating, i.e., selecting/deselecting, the transducer interface (36, 46) within the node,
the number of signal paths (83x, 86x) between each node (33, 43) equals the number of contact pads (81), and thus the electrical connections (6), in the first and second connector segments (31, 41, 42, 51, 52), and/or
at least one of the signal paths (83x, 86x) may be used to verify the position of a selected node, wherein said verification may be superimposed with other operations, such as a digital clock (CLK), to reduce the total number of signal paths (83x, 86x).

The transducer interface (36, 46) comprises preferably at least one uni- or bidirectional switch and optionally a signal amplifier or signal filter, which allows increased signal sensitivity. This configuration of the nodes (33, 43) enables multiplexing of the signals. Thus, the number of transducers per substrate (1) can be increased drastically without increasing the number of specific signal paths within the signal layer (83, 86) of the FPCB (8) and thus the number of contact pads (81) by simply increasing the number of nodes preferably by increasing the number of modules or designing a new module with more nodes. As an example, one contact pad (81), which connects to one discrete signal path (83x, 86x), may be—due to the achieved multiplexing—in contact with 50 or even more transducers (33, 43) located on separate nodes. Furthermore, the D-type flip-flop (35, 45) of all nodes (33, 43) across all modules (3, 4) are preferably connected in a daisy-chain fashion to allow modifying the state of each of the D-type flip-flop (35, 45) using only two signals, i.e., a data signal (D₀) and a clock signal (CLK), which may preferably originate from the external control unit (7).

For certain applications, an additional signal may be used to verify the selected node position before activating any of the transducers. This can be achieved by placing a feedback resistor of unique value at each node (Rₙ, Rₙ₊₁)), which has one end connected to the DFF output (Dₙ) within the node and the other end to an external sensing connection (S_{FB}). By having an appropriate relationship between the resistor values, for e.g., a logarithmic series, it can be ensured that each node selection is associated with a unique electric signal (current/voltage) which can be measured by the external controller using methods commonly known in the art.

Furthermore, the feedback signal (S_{FB}) can be superimposed on other signals, such as the digital clock signal (CLK), to reduce the number of signal paths (83x, 86x). Since digital logic circuits typically have a broad range of acceptable input voltage levels, the absolute values of the feedback resistors can be chosen such that any impact—due to the superimposition—may fall within those acceptable levels.

It is noted that each node (33, 43) may comprise multiple transducer interfaces (36, 46) and transducers (34, 44) in parallel which are controlled from a single DFF (35, 45). In this case, the transducer interfaces (36, 46) and transducers (34, 44) will typically connect/disconnect simultaneously. Hence the parallel transducer interfaces (36, 46) will require multiple parallel connections.

The D-type flip-flop (DFF; 35, 45), the transducer interfaces (36, 46) and the selectable transducer interface (STI) are most typically mounted onto the nodes (33, 43) of the modules (3, 4), and thus onto the FPCB (8) of the substrate (1).

Furthermore, the nodes (33, 43) of the modules (3, 4) may further comprise
further components such as resistors, capacitors, and application specific integrated circuits (ASIC), wherein the ASIC may be mounted onto the module before the most proximal module to convert all the signals, e.g., the signal emitted from the transducer interfaces (36, 46) or the D-type flip-flop (DFF; 35, 45) signals D₀ and CLK, from single ended to differential mode to improve noise suppression.

### Transducers (34, 44)

The term transducer (34, 44) refers according to the present invention to a device that converts energy, from one form into another form. Preferably, it converts input energy of one form into output energy of another form. Suitable transducers (34, 44) include sensors, i.e., a device that produces an output signal for the purpose of sensing a physical phenomenon.

A typical energy may be an electrical signal, e.g., a current or voltage, which is converted to or from, e.g., electromagnetic energy such as light or radio frequency (RF) waves; kinetic energy e.g., in piezos and accelerometers; a force, e.g., in pressure, touch or stretch sensors; or a chemical signal, e.g., electrically controlled drug release, or electrodes to measure an ionic strength such a pH, K⁺ or Na⁺.

In a particularly preferred embodiment, the transducers (34, 44) are sensors, in particular passive or active sensors, or actuators, wherein the transducers (34, 44) may be
in the form of a discrete component mounted on a surface of the FPCB (8) and connected to contact paths (83x, 86x). Thus, the type of transducers (34, 44) is not limited and thus any transducer (34, 44) may be mounted which is suitable for FPCB's;
or in the form of a pad on the external surface of the FPCB (8) connected with a contact path (83x, 86x) through a via. This provides fully integrated transducers (34, 44) not capable of any mal-production nor breakage of a connection, e.g., during wrapping the substrate (1) comprising the transducers (34, 44) around the catheter tube or cable (2a) and/or upon inserting the substrate (1) into the catheter tube (2a).

The size of the transducers (34, 44) may have a length and width of e.g., up to 2 mm and a height of e.g., up to 1 mm, preferably up to 0.5 mm. When the FPCB (8) is mounted onto the surface of the catheter tube or cable (2a), the transducers in the form of discrete components are most typically mounted onto the surface of the modules (3, 4)—and thus on the surface of the FPCB (8) which will be in contact with the surface of the catheter tube or cable (2a). And when the FPCB (8) is inserted into the catheter tube (2a), the transducers in the form of discrete components are most typically mounted onto the side of the FPCB (8) which will be directed towards the inside of the catheter tube (2a). Alternatively—or in addition—a portion of the transducer may be directed towards outside of the catheter tube or cable (2a) and thus exposed to external signals by e.g., an aperture in the FPCB (8). Or the transducers may be integrated on the surface of the FPCB (8), which is opposite the outside surface of the catheter tube or cable (2a), e.g., in the form of a pad linked to an individual signal path (83x, 86x) on an inner layer of the FPCB (8) through a via. Suitable external transducers (34, 44) are known to the skilled person in the art and commercially available. Vias and apertures, as well as the making of them in FPCB's are known to the skilled person in the art and described, e.g., in https://macrofab.com/blog/via-in-pad-pcb-design/. Suitable apertures are known to the skilled person in the art and are known under the terms “non plated though hole (NPTH)” and “plated though hole” (PTH). They may allow electrical pathways between two or more layers. Furthermore, vias may be filled to avoid any material transmission through them.

The transducers (34, 44) in the form of sensors convert an input signal to an electrical signal as output signal, which can be measured by a standard electronic device. Active sensors generate a measurable voltage or current in accordance with the external signal without the need of external electrical power supply. Non-limiting examples of active sensors include electrodes, photo diodes, thermo-couples, and piezo-electric transducers. Passive sensors, change their electrical property such as resistance or capacitance in accordance with the external signal, hence they require external electrical power to generate a measurable current or voltage. Non-limiting examples of passive sensors include piezo-resistors, photo-transistors, Resistance Temperature Detector (RTD) and strain gauges.

Some sensing mechanisms require application of a small excitation signal, such as ultrasonic or electric or electromagnetic waves, for the sensor to generate a response. Such excitation signal may be applied by the same transducer (34, 44) as the sensor, e.g., piezo-electric transducers, different transducer but same or similar in construction, e.g., electrodes, or a transducer of different form, e.g., light emitting diodes (LEDs).

Electrical signals generated by the sensor output, are sent via the FPCB (8) to the external control unit (7) for further processing and/or recording. Non-limiting examples of such data include bio-signals such as pH of the adjacent environment, e.g., blood; pressure, e.g., blood pressure or the contact pressure of the catheter (2) to adjacent human tissue; concentration of elements, ions or gases, e.g., potassium or oxygen, which are dissolved e.g., in a liquid such as blood; biopotentials, such as bio-signals from electrocardiogramalectromyography (EMG), electroencephalography (EEG); excitation based sensing such as photo-plethysmography, impedance, ultrasonic/optical imaging; and/or temperature of the environment. The recorded signal may be further used for diagnostic purposes or for general monitoring of the patient.

Actuators, such as electrodes or ultrasonic transducers, can also be used to generate a physiological change in the surrounding tissue for therapeutic reasons. Such actuators typically require more electrical power than required for diagnostic/monitoring functionality but have similar working principles. Non-limiting examples of therapeutic functionalities achieved using actuators are neural/muscular stimulation, e.g., using electrical and/or ultrasonic waves, neural suppression, e.g., again using electrical and/or ultrasonic waves, and/or tissue ablation, e.g., using radio-frequency alternating current.

The discrete transducers (34, 44) are preferably mounted onto one surface of the FPCB (8), wherein said surface is preferably the surface which is same as the one comprising the electronic components such as the D-type flip-flop (DFF; 35, 45) and the two or more transducer interfaces (36, 46). The transducers (34, 44) may be connected to the FPCB (8) by e.g., soldering, or using conductive glue, or any other methods known to the skilled person in the art, such as e.g., ACA or ACF.

### The FPCB (8)

The term flexible printed circuit board (FPCB, 8) is a layered composite material comprising electrically conductive and non-conductive, i.e., dielectric, layers. The conductive layers may be based on conductive metals and/or conductive organic polymers. The dielectric layers are most typically based on organic polymers. The thickness of the FPCB (8) may be in total—measured according to DIN 50986—may vary from 0.05 mm to 4 mm, preferably from 0.15 to 2 mm. Both, the thin material layers in combination with the type of materials lead to highly flexible and bendable, electrically conductive material.

The modules (3, 4, 5) with the connector segments (41, 42, 51, 52), and the nodes (33, 43) are based on the flexible printed circuit board FPCB (8). As such, the substrate (1) is based on the FPCB (8). Due to the complex inner structure of the FPCB (8), and thus the modules (3, 4, 5), with the multitude of interconnections, which are independent of the number of contact pads (81), neither the FPCB (8) nor the modules (3, 4, 5) or the contact pads (81) can be considered to be a cable.

On both broader surfaces, i.e., on the outer and inner side of the FPCB (8), it is possible to mount a number of components, e.g., transducers onto the outer surface, i.e., on the surface which will be opposite to the surface of the catheter tube or cable (2a), when the FPCB (8) is wrapped around the latter, and electronic components like the D-type flip-flop (DFF; 35, 45), transducer interfaces (36, 46) such as the selectable transducer interfaces (STI) are mounted typically onto the inner side, i.e., opposite to the side where the transducers (34, 44) are assembled.

In a particularly preferred embodiment, the flexible printed circuit board FPCB (8) is a layered composite material comprising:
contact pads (81) on the surface of the FPCB (8), wherein the contact pads (81) are
located on each connector segment (31, 41, 42, 51, 52), wherein said contact pads (81) are electrically connected to the contact pads (81) of the connector segment (31, 41, 42, 51, 52) of the adjacent modules (3, 4, 5), and
optionally on the surface of the nodes (33, 43) to function as transducer (34, 44),
a first dielectric layer (82),
a, i.e., one or more, signal layer (83),
optionally an adhesive layer (84),
optionally one or more sequences of stacked layers comprising a further dielectric layer (85), a further signal layer (86), an optional solder mask layer (87), and
optionally a dielectric coating (88),
wherein each signal layer (83, 86) comprises a multitude of individual signal paths (83x, 86x), wherein the contact pads (81) of each connector segment (31, 41, 42, 51, 52) are connected to an individual signal path (83x, 86x), wherein preferably each contact pad (81) is connected to the individual signal path (83x, 86x) through a via (813). This leads to highly versatile and highly flexible modules (3, 4, 5) and thus substrate (1), which is most suited to be wrapped around a catheter tube or cable (2a) and/or inserting the substrate (1) into the catheter tube (2a) to obtain the catheter (2) of the process of the invention. The signal paths (83x, 86x) of the FPCB (8), and thus of the modules (4, 5), may be all connected to each other.

In another preferred embodiment,
the contact pads (81) and signal layers (83, 86) are based on Cu, Au, Ni, Cr, Pd, Al, Ag, Sn, Pt, Ir—Pt and/or electrically conductive polymers such as PEDOT, Ag-PDMS, or electronic inks such as conductive silver inks, wherein the layers (81, 83, 86) may be based on the same or a different material,
the dielectric layers (82, 85) are based on a liquid-crystal polymer (LCP) such as ULTRALAM® 3850HT, polyimide (PI), and/or polyethylene terephthalate (PET),
the adhesive layer (84) is based on a liquid-crystal polymer (LCP) such as ULTRALAM® 3908, Polyimide (PI), polyethylene terephthalate (PET), and/or Dycoplast, the solder mask layer (87) is based on a liquid photopolymer, and/or a solder resisting epoxy polymer, and/or
the optional dielectric coating (88) is a single- or multi-component coating based on Parylene C, SiO2 and ceramic, or is based on a Parylene-type material, in particular Parylene C, Parylene D, Parylene HT or Parylene N.

These specifics lead to a particular advantageous embodiment of the FPCB (8).

The contact pads (81) may be laser-machined and/or coated to provide a 3-D pattern for a better electrical contact with the contact pads (81) of the adjacent connector segment (31, 41, 42, 51, 52), as well as to obtain an improved contact to skin and thus to obtain even a better signal quality, e.g., an improved signal-to-noise ratio or biocompatibility. Non-limiting, suitable materials to coat the layer (51) are electrically conductive materials and include silver (Ag), gold (Au), copper (Cu), electroless nickel immersion gold (ENIG), iridium-platinum (Ir—Pt), iridium dioxide (IrO2), titanium nitride (TiN), and/or polymers such as poly-3,4-ethylendioxythiophen (PEDOT) and silver-polydimethylsiloxane (Ag-PDMS). The thickness of such a coating may be between 0.05 μm and 1 μm, measured with X-ray according to DIN ISO 3497 or—if unsuitable for the specific case—scanning electron microscopy (SEM). The skilled person can make the proper selection.

The thickness of the layer (81) is preferably between 5 μm and 50 μm, in particular between 10 μm and 30 μm. The thickness of the layer (83) is preferably between 5 μm and 50 μm, in particular between 5 μm and 20 μm. And the thickness of the optional layer (86) is preferably between 5 μm and 50 μm, in particular between 10 μm and 40 μm, measured with X-ray according to DIN ISO 3497 or—if unsuitable for the specific case—scanning electron microscopy (SEM).

The first and the further dielectric layers (82, 85) separate the conductive layers (81, 83, 86) from each other. Suitable dielectric materials for the layers (81, 83, 86) are known to the skilled person. Non-limiting, but preferred materials for the dielectric layers (82, 85) include liquid-crystal polymer (LCP) and/or polyimide (PI). LCP—as example—provides a number of advantageous properties, including biocompatibility, high mechanical flexibility and strength, good dielectric characteristics, multilayer circuit capabilities, high compatibility to the solder mask material, high durability, very low water absorption, excellent high-frequency electrical properties and thus it is suitable for RF applications and is chemically inert. Furthermore, LCP allows to cut out arbitrary forms from a sheet of FPCB, e.g., by laser cutting.

The thickness of the layer (82) is preferably between 10 μm and 200 μm, in particular between 25 μm and 100 μm, and the thickness of the layer (85) is preferably between 5 μm and 50 μm, in particular between 5 μm and 20 μm, measured with X-ray according to DIN ISO 3497 or—if unsuitable for the specific case—scanning electron microscopy (SEM). The skilled person can make the proper selection.

The optional adhesive layer (84) adheres typically a conductive layer (81, 83, 86) to a dielectric layer (82, 85). Depending on the specifically used conductive layers (81, 83, 86) and dielectric layers (82, 85) and/or the process to manufacture the FPCB, the adhesive layer (84) might be omitted. Suitable adhesives for the layer (84) are commercially available and known to the skilled person in the art. A non-limiting, but preferred adhesive includes ULTRALAM®, in particular ULTRALAM® 3908, or Dycoplast. A typical thickness of the layer (84) ranges preferably between 5 μm and 50 μm, in particular between 10 μm and 40 μm, measured with X-ray according to DIN ISO 3497 or—if unsuitable for the specific case—scanning electron microscopy (SEM). The skilled person can make the proper selection.

The optional solder mask layer (87) may form the final layer of the FPCB (8) and thus covers and protects the layer underneath. In case the latter is e.g., a dielectric layer (82, 85) with sufficient resistance to the environment, the layer (87) may be omitted. Suitable materials for the layer (87) are commercially available and known to the skilled person in the art. He also can make the best selection. A typical thickness of the layer (87) ranges preferably between 5 μm and 50 μm, in particular between 10 μm and 40 μm, measured with X-ray according to DIN ISO 3497 or—if unsuitable for the specific case—scanning electron microscopy (SEM). It is often preferred that the solder mask layer (87) is not in direct contact with the outside environment, i.e., with the body liquid or tissue of the human body. The main purpose the layer (87) is to avoid electrical shorts while attaching the components such as transducers (34, 44), the activatable transducer interface (36, 46), the activation logic circuit such as the D-type flip-flop (DFF; 35, 45), optional integrated circuits (IC's), and connectors.

The Parylene-type materials of the optional dielectric coating (88) are inert, hydrophobic polymeric coating materials based on Poly-p-xylylene, and/or halogenated polymers thereof, wherein Parylene C is particularly preferred. Dielectric coating (88), such as Parylene-type materials and coatings based thereupon, are known to the skilled person in the art.

### The Process

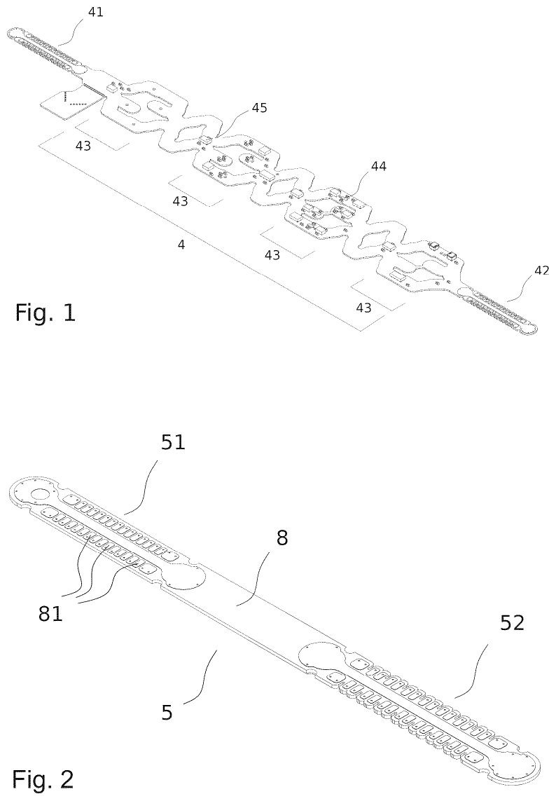

The modular sensing and actuating layered substrate (1) according to the invention is most preferably manufactured according to the process of the invention, wherein the process comprises
cutting out—preferably by laser-cutting—of a pre-shaped FPCB sheet a total of two or more of one or more active modules (4) comprising a first (41) and second (42) connector segment, and at least one node (43), optionally one or more spacer modules (5) comprising a first (51) and second (52) connector segment, and optionally the distal module (3) comprising the connector segment (31), and optionally at least one node (33);
mounting on each node (33, 43) of the one or more active modules (4), and of the optional distal module (3), the transducers (34, 44) onto at least one surface of the FPCB (8) and optionally the activatable transducer interface (36, 46) and the activation logic circuit comprising preferably the D-type flip-flop (DFF; 35, 45). An exemplary architecture of mounting the D-type flip-flop (35, 45), the transducer interfaces (36, 46) comprising a uni- or bidirectional switch and the transducers (34, 44) onto at least one surface of the FPCB (8) is shown in FIG. 1;
connecting the first connector segment (41, 51) of the active (4) and the optional spacer (5) modules, and the connector segment (31) of the optional distal module (3), with the second connector segment (42, 52) of the adjacent active (4) or spacer (5) module via a flat, i.e., large, surface of the connector segments (31, 41, 42, 51, 52), thus forming the substrate (1) of at least one the claims 1 to 11;
wrapping the obtained substrate (1) around a catheter tube or cable (2a) and/or inserting the substrate (1) into the catheter tube (2a), wherein the substrate (1) is adhered to the catheter tube of cable (2a) to obtain the electronic catheter (2). The step of wrapping around or inserting into a catheter tube or cable (2a) is known to the skilled person in the art. And the step of adhering the substrate (1) to the catheter tube or cable (2a) is preferable made by thermowelding, thermocompression bonding and/or means of an adhesive. Uncoated catheter tubes (2a) are state-of-the-art catheter tubes and well known to the skilled person in the art.

Upon connecting the first connector segment (31, 41, 51) to the second connector segment (42, 52) of the modules (3, 4, 5) via the flat, i.e., large, surface of the connector segments (31, 41, 42, 51, 52), an electrical connection (6) may be placed between the contact pads (81), between the areas besides the contact pads (81) of the first and second connector segments (31, 41, 42, 51, 52), and/or the electrical connection (6) is obtained by a layer which surrounds the first and the adjacent second connector segments (31, 41, 42, 51, 52).

In case at least one of the modules (3, 4, 5) contains one or more additional connection pads for testing the interconnected modules in parallel during the manufacturing process, the working of the individual modules—or even of the assembled substrate (1)—is verified and, in case proper working is proven, said connection pads are removed, i.e., detached, physically, e.g., by cutting. This optional step is preferably performed before joining the substrate (1) with the catheter tube (or cable (2a), i.e., before wrapping the substrate (1) around the catheter tube or cable (2a) and/or inserting the substrate (1) into the catheter tube (2a).

It is noted that besides the transducers (34, 44) and the optional activatable transducer interface (36, 46) and the optional activation logic circuit which preferably comprises the D-type flip-flop (DFF; 35, 45), further components may be added. Non-limited examples of such components include resistors, capacitors, and application specific integrated circuits, i.e., ASIC, wherein the ASIC may be mounted onto the module before the most proximal module to convert all the signals, e.g., the signal emitted from the transducer interfaces (36, 46) and/or from the DFF (35, 45) from single ended to differential mode to improve noise suppression.

In one embodiment of the process of the invention, the pre-shaped FPCB sheet is made by:
crimping the first dielectric layer (82), the signal layer (83), the optional adhesive layer (84) and the further dielectric layer (85), wherein the dispensable portions of the signal layer (83) are etched away to generate the signal paths (83x) prior to apply the first dielectric layer (82). Thus, the desired architecture of the specific layers of the individual modules (3, 4, 5) with the connector segments (31, 41, 42, 51, 52) and the nodes (33, 43) can individually be made according to the specific needs; and
generating the vias (813) by boring or lasering through at least the first dielectric layer (82) to the signal layer (83), filling the vias (813), e.g., with the same material of the signal layer (83), and plate through to connect the signal layer (83) with the thus formed contact pads (81).

In a preferred embodiment of the process of the invention, connecting the first (31, 41, 51) with the second (42, 52) connector segment is obtained by placing the first or second connector segment (31, 41, 51; 42, 52) next to the second or first connector segment (42, 52; 31, 41, 51) of the modules (3, 4, 5) and placing
the connection i) between at least one, preferably all, of the contact pads (81) of the first connector segment (31, 41, 51) and at least one, preferably all, of the contact pads (81) of the second connector segment (42, 52),
the connection ii) between the area besides to the contact pads (81) of the first connector segment (31, 41, 51) and the area besides to the contact pads (81) of the second connector segment (42, 52), and/or
surrounding the first connector segment (31, 41, 51) and the adjacent second connector segment (42, 52) with the layer of connection iii).

This provides a good, basically permanent electrical connection between the pads (81) which maintains flexibility of the substrate (1) required to form the catheter (2). Furthermore, conducting the electrical current in a most optimal manner is maintained.

In another preferred embodiment of the process, the catheter tube or cable (2a) is based on a thermoplastic polymer, polyurethane (PU), polyolefin, polytetrafluoroethylene (PTFE), and/or polydimethylsiloxane (PDMS). Hence, the catheter tube of cable (2a) is highly biocompatible. And since the substrate (1) being based on FPCB (8) is biocompatible as well, the electronic catheter (2) according to the process of the invention is also highly biocompatible.

In an even further preferred embodiment, the
catheter tube or cable (2a) has an outer diameter from 2 to 10 mm, preferably from 2.5 to 7.5 mm, in particular from 3 to 5 mm, determined according to DIN 50986;
the substrate (1) being wrapped around the catheter tube or cable (2a) has a thickness from 0.05 to 4 mm, preferably from 0.15 to 2 mm, determined according to DIN 50986; and/or
the catheter (2) comprising the catheter tube or cable (2a) and the substrate (1) wrapped around the catheter tube or cable (2a) has a diameter from 2 to 18 mm, preferably from 2.8 to 9 mm, determined according to DIN 50986.

### LIST OF REFERENCE SIGNS

1 Modular layered substrate (1)
2 electronic catheters (2) comprising the substrate (1)
   2acatheter tube or cable (2a)
3 distal module (3)
   31 connector segment (31) of distal module (3)
   33 node (33) of distal module (3)
   34 transducers (34) of distal module (3)
   35 D-type flip-flop (DFF) of distal module (3)
   36 transducer interfaces (36) of distal module (3)
4 active modules (4)
   41 first (41) connector segment of module (4)
   42 second (42) connector segment of module (4)
   42 node (43) of module (4)
   43 transducer (44) of module (4)
   44 D-type flip-flop (DFF) of active module (4)
   45 transducer interfaces (46) of active module (4)
5 spacer modules (5)
   51 first (51) connector segment of module (5)
   51 second (52) connector segment of module (5)
6 electrical connection (6)
7 external control unit (7)
8 Flexible Printed Circuit Board FPCB (8)
   81 contact pads (81) of the FPCB (8)
   813 vias (813)
   82 first dielectric layer (82) of the FPCB (8)
   83 signal layer (83) of the FPCB (8)
   83x individual signal path of signal layer (83)
   84 optional adhesive layer (84) of the FPCB (8)
   85 optional further dielectric layer (85) of the FPCB (8)
   86 optional further signal layer (86) of the FPCB (8)
   86x individual signal path of signal layer (86)
   87 optional solder mask layer (87) of the FPCB (8)
   88 optional dielectric coating (88) of the FPCB

The following figures present non-limiting embodiments, which are not restricting or narrowing the invention. These explanations are part of the description:

FIG. 1 discloses a non-limiting example of the active module (4), which comprises a first (41) and a second (42) connector segment, and at least one node (43), wherein each node (43) comprises a number of transducers (44) and an activation logic circuit comprising a D-type flip-flop (DFF, 45) for activating/deactivating the transducer interface (46, not shown). The active module (4) is based on a flexible printed circuit board FPCB (8), and the nodes (43) are formed to provide a broadened shape to allow placing the transducers at several places along the periphery of the catheter for sensing/actuating in multiple directions and optionally further components onto the node (43). Furthermore, the broadened shape of the node (43) increases flexibility of the node (43), and finally of the substrate (1). The form of the shape of the nodes (43) may be arbitrary, but preferably optimized to the specific needs of the substrate (1) and thus the catheter (2) comprising the substrate (1). The nodes (43) are connected to the adjacent nodes by an FPCB (8) segment, e.g., of narrower width.

The nodes (43) are arranged in a linear manner forming the active module (4), which is terminated on each end by a connector segment (41, 42), which allow connecting the active module (4) with another active module (4), with the distal module (3) and/or with an optional spacer module (5).

Between the first connector segment (41) and the adjacent node (43) is on one side an optional detachable segment arranged, which is also based on the same FPCB (8). Such a detachable segment allows to check whether or not all connections are functioning. After having made such a performance check, the detachable segments may be detached, e.g., with pincers.

The optional distal module (3) may be of the same principal design as the active module (4), wherein the distal module (3) comprises only one connector segment (31), instead of two as the active module (4).

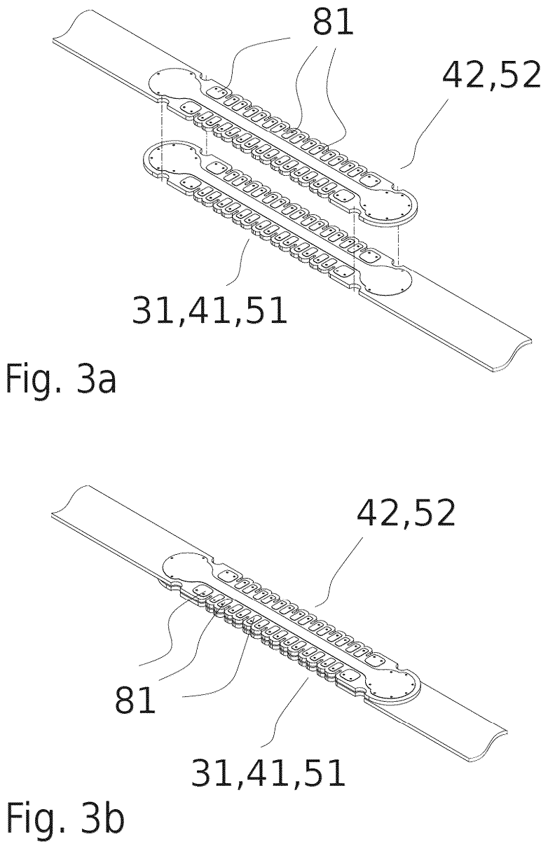

FIG. 2 discloses a non-limiting example of an optional spacer module (5). The module (5) comprises a first (51) and a second (52) connector segment, but no node nor a transducer. The connector segments (51, 52) are having the same structure as the connector segments (41, 42) of the active module (4) and the connector segment (31) of the optional distal module (3) to provide a large flexibility in assembling the modules (4, 5) in any order and terminating the assembled substrate (2) by the optional distal module (3).

The illustration of the first (51) and the second (52) connector segment is different to show different embodiments, although the number and position of the contact pads (81) of the FPCB (8) are the same for each connector segment (51, 52). While the edges of the FPCB (8) or the first connector segment (51) are straight to increase the stiffness of the connector segment (51), the edges of the second connector segment (52) are patterned, i.e., between the contact pads (81) is a recess to increase flexibility of the connector segments (31, 41, 51, 42, 52).

Furthermore, both connector segments (51, 52) are equipped with an optional reinforcement element having a bone-like shape to avoid breakage of the connector segments (51, 52). The end of the first connector segment (51), including the reinforcement element, is equipped with a hole to allow to fix e.g., a thread to pull the spacer (5) or any other module (3, 4) through a catheter tube (2a) and/or through a hole in the catheter tube (2a).

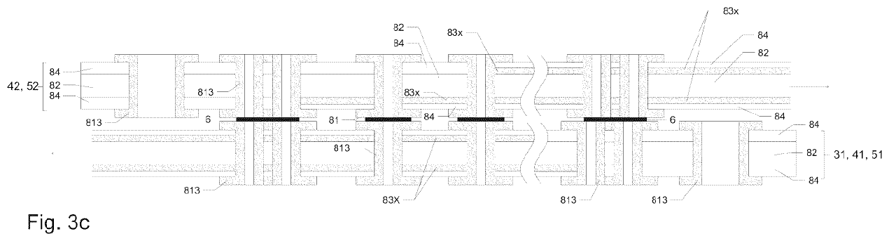

FIG. 3a shows a first connector segment (31, 41, 51) of either the optional distal module (3), the active module (4) or the optional spacer module (5), and a second connector segment (42, 52) of either the active module (4) or the optional spacer module (5).

The connector segments (31, 41, 51, 42, 52) contain a multiple of electrically conductive contact pads (81) to provide a proper electrical connection (6) between the contact pads (81) of the first (31, 41, 51) and the second (42, 52) connector segments, when connected.

Each connector segment (31, 41, 51, 42, 52) contains exemplary an optional reinforcement element having a bone-like shape to avoid breakage. Furthermore, the edge of the FPCB (8) of the connector segments (31, 41, 51, 42, 52) is optionally patterned, i.e., between contact pads (81) is a recess to increase flexibility of the connector segments (31, 41, 51, 42, 52).

FIG. 3b shows the first connector segment (31, 41, 51) connected to the second connector segment (42, 52) via one of their flat, i.e., large, surfaces, i.e., the surface having the larger extension, wherein the electrically conductive contact pads (81) of the first connector segment (31, 41, 51) are properly electrically connected to the contact pads (81) of the second connector segment (42, 52). Thus, a proper electrical connection (6) between the first (31, 41, 51) and second (42, 52) connector segments is obtained.

FIG. 3c shows a cross section of the first connector segment (31, 41, 51) connected to the second connector segment (42, 52) of the connected modules (3, 4, 5) as shown in FIG. 3b.

The first and second connector segments (31, 41, 42, 51, 52) are electrically connected to each other via electrical connections (6), which are placed between the contact pads (81) of the first (lower) and the second (upper) segment. The arrows to the left (lower first) and right (upper second) segment indicate the direction of the continuation of the modules (3, 4, 5).

The contact pads (81) are exemplary connected to the individual signal path (83x, 86x) through vias (813).

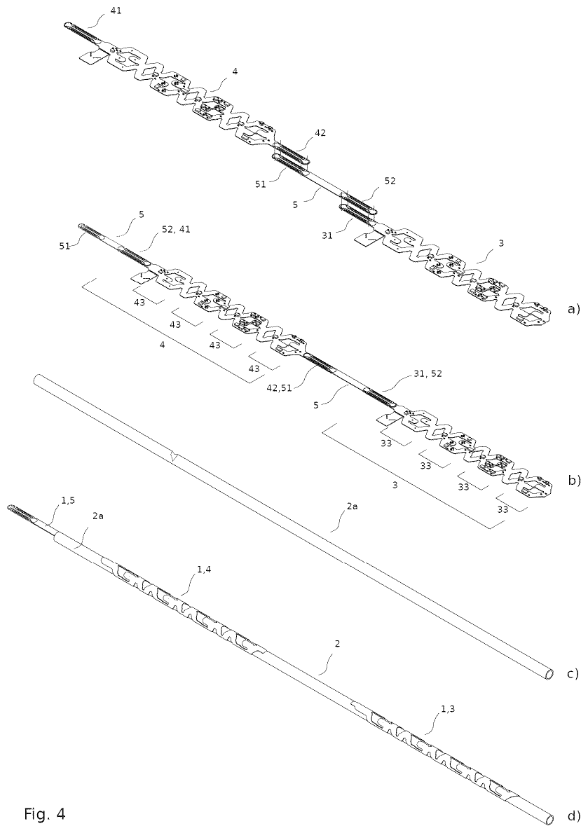

FIG. 4 presents the various steps from individual modules (3, 4, 5; FIG. 4a) to the assembled substrate (1, FIG. 4b) being wrapped around a catheter tube or cable (2a, FIG. 4c) to the manufactured electronic catheter (2, FIG. 4d). It visualizes the order, type and number of the different modules (4, 5) can easily be varied, with or without the presence of a distal module (3) terminating the order. Hence, a highly customizable electronic catheter (2) can be obtained.

FIG. 4a shows specifically—from right to left—the optional distal module (3) with one connector segment (31) and an optional detachable segment for performance test; an optional spacer module (5) with the first (51) and second (52) connector segment; an active module (4) with the first (41) and second (42) connector segment and an optional detachable segment for performance test; followed by another optional spacer module (5). Both, the optional distal module (3) as well as the active module (4) comprise a multitude of nodes (33, 43) having the same or a different architecture, a number of transducers (44) and an activation logic circuit comprising a D-type flip-flop (DFF, 45) for activating/deactivating the transducer interface (46, not shown).

The connector segments (52/31 and 42/51) are placed above each other to indicate that they will be electrically connected to each other and thus forming the substrate (1) according to the invention.

FIG. 4b shows the electrically connected modules (3, 4, 5) of FIG. 4a forming the substrate (1) according to the invention. The exemplary order of the modules is 3-5-4-5, but it could be prolonged based on the specific needs of the catheter (2).

FIG. 4c shows an example of a catheter tube (2a) with an optional hole towards to proximal, i.e., left, end of the tube (2a) to allow a connector segment (5) to pass from the outer surface of the tube (2a) into the inner area of the tube (2a).

FIG. 4d shows the finally assembled electronic catheter (2) comprising the substrate (1) of FIG. 4b with the distal module (3), a spacer module (5), an active module (4) and another spacer module (5) leading towards the external controller (7, not shown).

The spacer module (5) on the left side of the catheter (2) is passed through the hole visualized in FIG. 4c and thus it is embedded inside of the catheter tube (2a).

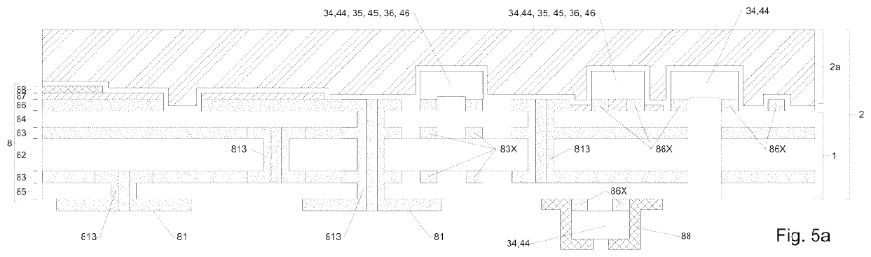

FIG. 5a shows a schematic cross-section representing various possible scenarios of sections of the electronic catheter (2) with a portion of the FPCB (8) of a node (33, 43) of either the distal (3) or active (4) module. The FPCB (8) is wrapped around the outer surface of the catheter tube or cable (2a), hence attached, i.e., adhered, onto its surface, e.g., by means of thermocompression bonding, thermowelding and/or with an adhesive. Thus, the lower side of FIG. 5a represents the outer, i.e., exterior, side of the electronic catheter (2), which will be—when in use—in contact with liquid or tissue of the human body.

The exemplified FPCB (8), being a layered composite material, comprises a first dielectric layer (82), which separates two signal layers (83). The one signal layer (83) being arranged towards the catheter tube or cable (2a) is adhered with the optional adhesive layer (84) to a further signal layer (86), which itself is covered by the optional solder mask layer (87). The latter is partially coated by the optional dielectric coating (88), which is adhered to the surface of the catheter tube or cable (2a). The illustrated white layer between the tube or cable (2a) and the different layers of the FPCB (8) represents the interface between the tube or cable (2a) and the FPCB (8).

The via (813) on the left side of FIG. 5a connects one signal layer (83) with the surface of the FPCB (8), on which a contact pad (81) is arranged. The via (813) is filled with e.g., the same material as the signal layer (83), such as gold.

The next via (813) in FIG. 5a, which is also filled, e.g., with gold, connects the two signal layers (83) with each other. The adjacent via (813) to the right connects the contact pad (81) on the surface of the FPCB (8) with all three signal layers (83, 86). And the most right via (831) connects the three signal layers (83, 86) with each other.

The signal layers (83, 86) may be—e.g., in a certain area of the FPCB (8)—in the form of a flat layer, and/or—e.g., in other areas of the FPCB (8)—in the form of a number of individual signal paths (83x, 86x).

On the surface of the FPCB (8), which is affixed to the catheter tube or cable (2a), are electronic components, such as a transducer (34, 44), a D-type flip-flop (DFF, 35, 45), and a transducer interface (36, 46) mounted and connected to signal paths (86x). The FPCB (8) is at the position of the most right transducer (34, 44) interrupted to provide a void from the transducer (34, 44) towards the outside surface of the FPCB (8).

On the outer, i.e., exterior, surface of the FPCB (8), which is opposite to the catheter tube or cable (2a), is another transducer (34, 44) mounted and connected to signal paths (86x). Most of said transducer (34, 44) is coated with the optional dielectric coating (88).

FIG. 5b shows—similar to FIG. 5a—a schematic cross-section representing various possible scenarios of sections of the electronic catheter (2) with a portion of the same FPCB (8) of a node (33, 43) of either the distal (3) or active (4) module. The FPCB (8), however, is inserted into the catheter tube (2a), and attached, i.e., adhered, onto the inner surface of the tube, e.g., by means of thermocompression bonding, thermowelding and/or with an adhesive. Thus, the upper side of FIG. 5b, i.e., the upper side of the FPCB (8), is directed towards the center, and thus is arranged in the interior, of the catheter tube (2a). The lower side of FIG. 5b, i.e., of the catheter tube (2a), is directed—when in use—towards the liquid or tissue of the human body.

The substrate (1) of FIG. 5b is—except for the added dielectric coating (88) over the electronic components (34, 44, 35, 45, 36, 46) and the via (813) on the upper side of the FPCB (8) and the omitted dielectric coating (88) over the transducer (34, 44) being mostly covered by the catheter tube (2a), basically the same as the one of FIG. 5a. This demonstrates that both sides of the substrates can be adhered to a surface of the catheter tube or cable (2a). This flexibility increases the versatility of the substrate (1).

The exemplary transducer (34, 44) on the most right side of FIG. 5b is mounted and connected to signal paths (86x). The FPCB (8) is at the position of the transducer (34, 44) interrupted to provide a void from the transducer (34, 44) towards the surface of the FPCB (8). Said void is positioned over a void, i.e., hole, in the catheter tube (2a) to allow that e.g., body liquid can reach the transducer (34, 44). The hole of the catheter tube (2a) may be made before or after adhering the substrate (1) to the surface of the catheter tube (2a), wherein the diameter of the hole of the tube (2a) may be larger than the diameter of the void of the FPCB (8).

For further details of FIG. 5b see the explanations of FIG. 5a.

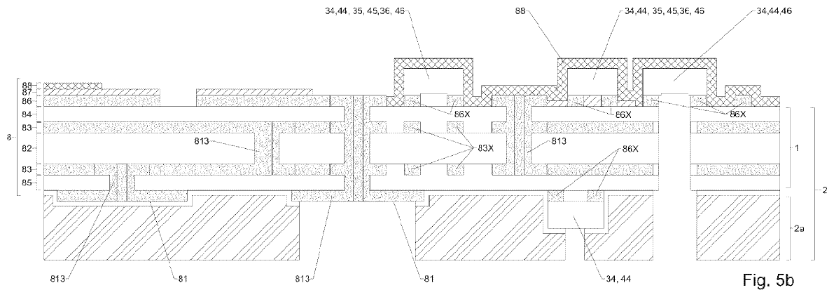

FIG. 6 presents a non-limiting example of a schematic section of the substrate (1) comprising two active modules (4) being connected to each other via a spacer module (5). The active module (4) on the left is further connect to another spacer module (5) which leads—either directly or via further modules (4, 5)—to the external controller (7).

The modules (4, 5) are connected to each other via the connector segments (42/51) and (52/41), respectively. All connector segments (41, 42, 51, 52) comprise the same number of electrically conductive contact pads (81) to allow a good electrical connection (6) between the connector segments (42/51) and (52/41), and thus between the modules (4, 5)—and therefore within the substrate (1) as a whole. Each contact pad (81) is connected to an individual signal path (83x, 86x). Hence, the contact pads (81) of the first (41, 51) and the second (42, 52) connector segments are connected to each other via said signal paths (83x, 86x).

Within the active modules (4) are a number of nodes (43) arranged and connected to the signal paths (83x, 86x).

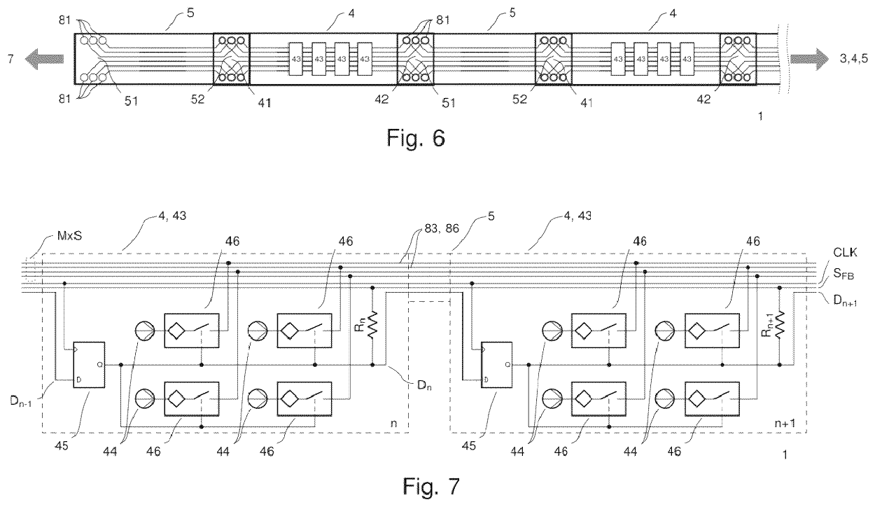

FIG. 7 presents an exemplary, non-limiting activation logic circuit with two nodes (43) at positions n and n+1, respectively, of two active modules (4) being connected to each other via a spacer module (5). Each node (43) comprises a D-type flip-flop (45) with a data signal from the previous node (Dₙ₋₁) and a clock signal (CLK). Furthermore, the DFF (45) output Dₙ is electrically connected to exemplary four transducer interfaces (46) which activates the connection between the transducers (44) and individual signal paths (83x, 86x) of the signal layer (83, 86) common for all the nodes. Dₙ signal also becomes the input for the next node (n+1). The Dₙ signal may also be connected to a sensing connection signal S_{FB} via a node feedback resistor (Rₙ). This arrangement allows activating only the transducer interfaces (46) in a given node (n). Thus, the transducers (34, 44) signals from all the nodes (43) within the substrate (1)—despite their large number—can be acquired from only a few multiplexed signal paths (MxS) in a round-robin manner.

1. Modular sensing and actuating layered substrate (1) for electronic catheters (2), comprising a total of two or more of
one or more active modules (4), each module (4) comprising a first (41) and a second (42) connector segment, and at least one node (43) comprising one or more transducers (44), and
optionally one or more spacer modules (5), each module (5) comprising a first (51) and a second (52) connector segment, but no node nor transducer, wherein the first connector segments (41, 51) of the active (4) and the optional spacer (5) modules are electrically connected to the second connector segment (42, 52) of the adjacent active (4) or spacer (5) module, characterized in that
the modules (4, 5) with the connector segments (41, 42, 51, 52), and the nodes (43) are based on a flexible printed circuit board FPCB (8), and
each connector segment (41, 42, 51, 52) comprises on at least one flat, i.e., large, surface of the FPCB (8) a multiple of electrically conductive contact pads (81), wherein, when the first connector segment (41, 51) of one module (4, 5) is electrically connected via an electrical connection (6) to the second connector segments (42, 52) of another module (4, 5), the contact pads (81) of the first connector segment (41, 51) are in contact with the contact pads (81) of the second connector segment (42, 52).

2. Substrate (1) according to claim 1, characterized in that the substrate (1) further comprises a distal module (3) comprising one connector segment (31), and preferably at least one node (33) comprising one or more transducers (34), wherein the distal module (3) with the connector segment (31), and the nodes (33) are based on the flexible printed circuit board FPCB (8).

3. Substrate (1) according to claim 1 or 2, characterized in that the optional distal module (3), the one or more active modules (4) and the optional one or more spacer modules (5) are connected to form a longitudinal substrate (1) which is capable to be wrapped around the catheter (2), wherein the connector segment (41, 51) of the module (4, 5), which is furthest away from the distal module (3), or from the most opposite connector segment (42, 52), is configured to be connected to an external control unit (7).

4. Substrate (1) according to at least one of the claims 1 to 3, characterized in that the flexible printed circuit board FPCB (8) is a layered composite material comprising
contact pads (81) on the surface of the FPCB (8), wherein the contact pads (81) are
located on each connector segment (31, 41, 42, 51, 52), wherein said contact pads (81) are electrically connected to the contact pads (81) of the connector segment (31, 41, 42, 51, 52) of the adjacent modules (3, 4, 5), and
optionally on the surface of the nodes (33, 43) to function as transducer (34, 44),
a first dielectric layer (82),
a signal layer (83),
optionally an adhesive layer (84),
optionally one or more sequences of stacked layers comprising a further dielectric layer (85), a further signal layer (86), an optional solder mask layer (87), and
optionally a dielectric coating (88),
wherein each signal layer (83, 86) comprises a multitude of individual signal paths (83x, 86x), wherein the contact pads (81) of each connector segment (31, 41, 42, 51, 52) are connected to an individual signal path (83x, 86x), wherein preferably each contact pad (81) is connected to the individual signal path (83x, 86x) through a via (813).

5. Substrate (1) according to claim 4, characterized in that
the contact pads (81) and signal layers (83, 86) are based on Cu, Au, Ni, Cr, Pd, Al, Ag, Sn, Pt, Ir-Pt and/or electrically conductive polymers such as PEDOT, Ag-PDMS, or electronic inks such as conductive silver inks, wherein the layers (81, 83, 86) may be based on the same or a different material,
the dielectric layers (82, 85) are based on a liquid-crystal polymer (LCP) such as ULTRALAM® 3850HT, polyimide (PI), and/or polyethylene terephthalate (PET),
the adhesive layer (84) is based on liquid-crystal polymer (LCP) such as ULTRALAM® 3908, Polyimide (PI), polyethylene terephthalate (PET), and/or Dycoplast,
the solder mask layer (87) is based on a liquid photopolymer, and/or a solder resisting epoxy polymer, and/or
the optional dielectric coating (88) is based on a single- or multi-component coating based on Parylene C, SiO2 and ceramic, or is based on a Parylene-type material, in particular Parylene C, Parylene D, Parylene HT or Parylene N.

6. Substrate (1) according to at least one of the claims 1 to 5, characterized in that the electrical connection (6) between the first connector segment (31, 41, 51) of the modules (3, 4, 5) and the second connector segment (42, 52) of the adjacent module (4, 5) is a connection
i) between at least one, preferably all, of the contact pads (81) of the first connector segment (31, 41, 51) and at least one, preferably all, of the contact pads (81) of the second connector segment (42, 52),
ii) between the area besides to the contact pads (81) of the first connector segment (31, 41, 51) and the area besides to the contact pads (81) of the second connector segment (42, 52), and/or
iii) obtained by a layer which surrounds the first connector segment (31, 41, 51) and the adjacent second connector segment (42, 52),
wherein
the connection i) is preferably a solder, in particular a lead-free solder alloy such as Sn96.5Ag3Cu0.5; an electrically conductive adhesive, wherein said adhesive is preferably an isotropic or anisotropic adhesive; or a welding, preferably obtained by thermo-sonic compression;
the connection ii) is preferably a mechanical engagement; an engagement aided by magnets or a magnet and a magnetizable material; or based on an adhesive; and/or
the layer of connection iii) is preferably obtained by molding, spraying, wrapping and/or encasing the first and second connector segments (31, 41, 42, 51, 52), wherein said layer is preferably based on a non-conductive material.

7. Substrate (1) according to at least one of the claims 1 to 6, characterized in that each node (33, 43) of the distal (3) and active (4) modules comprises one or more transducers (34, 44), an activatable transducer interface (36, 46), and an activation logic circuit comprising preferably one or more D-type flip-flop (DFF, 35, 45) for activating/deactivating the transducer interface (36, 46) within the node (33, 43),
the number of signal paths (83x, 86x) between each node (33, 43) equals the number of contact pads (81) in the first and second connector segments (31, 41, 42, 51, 52) and/or
at least one of the signal paths (83x, 86x) may be used to verify the position of a selected node, wherein said verification may be superimposed with other operations, such as a digital clock (CLK), to reduce the total number of signal paths (83x, 86x).

8. Substrate (1) according to at least one of the claims 1 to 7, characterized in that the transducers (34, 44) are sensors or actuators, wherein the transducers (34, 44) may be
in the form of a discrete component mounted on a surface of the FPCB (8) and connected to contact paths (83x, 86x),
or in the form of a pad on the external surface of the FPCB (8) connected with a contact path (83x, 86x) through a via.

9. Substrate (1) according to at least one of the claims 1 to 8, characterized in that the connector segments (31, 41, 42, 51, 52) are identical in terms of number and location of contact pads (81) to allow each module (3, 4, 5) to be daisy-chained to any other module (4, 5), wherein at least one of the modules (3, 4, 5) may be interfaced by the external control unit (7) via the same electrical connections (6) as the module (4, 5) which is located most proximally to the external control unit (7).

10. Substrate (1) according to at least one of the claims 1 to 9, characterized in that
the active module (4) is connected to one or two other active modules (4), to one or two spacer modules (5), and/or to the distal module (3),
the spacer module (5) is connected to one or two active modules (4), and/or to the distal module (3), and/or
the distal module (3) is connected to the active module (4) or the spacer module (5),
wherein the connector segment (41, 51) of the module (4, 5) which is furthest away from the distal module (3), or from the most opposite connector segment (42, 52), is configured to be connected to the external control unit (7).

11. Substrate (1) according to at least one of the claims 1 to 10, characterized in that
the one or more active modules (4) and/or the optional distal module (3) comprise at least 1, preferably 2 to 50, in particular 2 to 20, nodes (33, 43), wherein, when the substrate (1) comprises a plurality of modules (4), the number of nodes (43) of each module (4) may be different, and the number and type of transducers (44) on each node (43) may be the same or different, and/or
the shape of the one or more active modules (4), of the optional one or more spacer modules (5), and of the optional distal module (3) exhibit a linear shape, a zig-zag shape, U-shape, Ω-shape, a meandering form, and/or is based on a polygonal, honeycomb or scaffolding shape.

12. Process to manufacture an electronic catheter (2) comprising the modular layered substrate (1) of at least one of the claims 1 to 11, comprising
cutting out of a pre-shaped FPCB sheet a total of two or more of one or more active modules (4) comprising a first (41) and second (42) connector segment, and at least one node (43), optionally one or more spacer modules (5) comprising a first (51) and second (52) connector segment, and optionally the distal module (3) comprising the connector segment (31), and optionally at least one node (33);
mounting on each node (33, 43) of the one or more active modules (4), and of the optional distal module (3), the transducers (34, 44) onto at least one surface of the FPCB (8) and optionally the activatable transducer interface (36, 46) and an activation logic circuit comprising preferably the D-type flip-flop (DFF; 35, 45);
connecting the first connector segment (41, 51) of the active (4) and the optional spacer (5) modules with the second connector segment (42, 52) of the adjacent active (4) or spacer (5) module via a flat, i.e., large, surface of the connector segments (31, 41, 42, 51, 52), thus forming the substrate (1) of at least one the claims 1 to 11;
wrapping the obtained substrate (1) around a catheter tube or cable (2a) and/or inserting the substrate (1) into the catheter tube (2a), wherein the substrate (1) is adhered to the catheter tube or cable (2a) to obtain the electronic catheter (2).

13. Process according to claim 12, characterized in that connecting the first (31, 41, 51) with the second (42, 52) connector segment is obtained by placing the first or second connector segment (31, 41, 51; 42, 52) next to the second or first connector segment (42, 52; 31, 41, 51) of the modules (3, 4, 5) and placing
the connection i) between at least one, preferably all, of the contact pads (81) of the first connector segment (31, 41, 51) and at least one, preferably all, of the contact pads (81) of the second connector segment (42, 52),
the connection ii) between the area besides to the contact pads (81) of the first connector segment (31, 41, 51) and the area besides to the contact pads (81) of the second connector segment (42, 52), and/or
surrounding the first connector segment (31, 41, 51) and the adjacent second connector segment (42, 52) with the layer of connection iii).

14. Process according to claim 12 or 13, characterized in that the catheter tube or cable (2a) is based on a thermoplastic polymer, polyurethane (PU), polyolefin, polytetrafluoroethylene (PTFE), and/or polydimethylsiloxane (PDMS).

15. Process according to at least one of the claims 12 to 14, characterized in that
catheter tube or cable (2a) has an outer diameter from 2 to 10 mm, preferably from 2.5 to 7.5 mm, in particular from 3 to 5 mm, determined according to DIN 50986;
the substrate (1) being wrapped around the catheter tube or cable (2a) has a thickness from 0.05 to 4 mm, preferably from 0.15 to 2 mm, determined according to DIN 50986; and/or
the catheter (2) comprising the catheter tube or cable (2a) and the substrate (1) wrapped around the catheter tube or cable (2a) has a diameter from 2 to 18 mm, preferably from 2.8 to 9 mm, determined according to DIN 50986.
